(19) United States
(12) Patent Application Publication
(10) Pub. No.: US 2022/0222835 A1
(43) Pub. Date: Jul. 14, 2022

(54) Endoscopic image registration

(71) Applicant: Xiaoning Huai, Sunnyvale, CA (US)

(72) Inventor: Xiaoning Huai, Sunnyvale, CA (US)

(73) Assignee: Real Image Technology Co., Ltd, Shenzhen (CN)

(21) Appl. No.: 17/706,666

(22) Filed: Mar. 29, 2022

Related U.S. Application Data
(63) Continuation-in-part of application No. 17/676,220, filed on Feb. 20, 2022.

(30) Foreign Application Priority Data
Feb. 6, 2022 (CN) 202220249273X

(51) Int. Cl. G06T 7/33; G06T 7/73; G06T 17/00; G06T 15/50; G06T 7/00; A61B 34/30; A61B 1/00; A61B 34/10; B33Y 30/00; B33Y 50/02; B33Y 10/00

(52) U.S. Cl. CPC G06T 7/344; A61B 2034/105; G06T 17/00; G06T 15/506; G06T 7/0014; A61B 34/30; A61B 1/00006; A61B 34/10; B33Y 30/00; B33Y 50/02; B33Y 10/00; G06T 2200/04; G06T 2200/08; G06T 2207/10028; G06T 2207/20221; G06T 2207/30004; G06T 2210/41; G06T 7/75

(57) ABSTRACT

Disclosed is a system and method for endoscopic image registration, based on a 3D spectral data model of a biological body part built on preoperative 3D imageries. Dynamic registration of the model is rendered in an endoscopy or a robotic surgery leveraging captured point clouds of surface of anatomy of the body part. Position of the body parts are tracked through the registration for navigating a surgical robot. The model masked with the point cloud is displayed so that change of the position of the body part could be easily perceived by the medical staff.

### TECHNICAL FIELD

The invention is applied to the fields of biology and medicine, and especially to medical image processing, endoscopic image registration and automatic surgical robots.

### BACKGROUND

Endoscopy used for minimally invasive or natural orifice inspection or surgery usually operates with spectral image data captured by a camera. CT, MRI, and ultrasound and other 3D imaging modalities are often used for preoperative planning or as intraoperative auxiliary data. Positions of a human body part at surgical site need to be tracked during a surgery to assist operation by a surgeon or to guide a surgical robot, wherein tracking is accomplished through fusion or registration of data from various imaging modalities. To date, prior art image registration is primarily biased on registering endoscopic images with 3D imageries obtained preoperatively or intraoperatively as reference target.

### SUMMARY OF THE INVENTION

The invention discloses an image processing system, comprising a data acquisition module, a processing module and a display module; the data acquisition module may obtain a model of three-dimensional spectral data, or the model and a point cloud, or the model, the point cloud, and other imagery than the point cloud of a body part; the processing module may perform one or more of the following: 3D printing the model; adjusting the model referencing the point cloud; 3D printing the adjusted model; performing a first registration of the adjusted model with reference to the point cloud; 3D printing the registered model; controlling a surgical robot or an endoscope with reference to the first registration; obtaining one or more of fusions of two or more of the model, the point cloud, and the other imagery than the point cloud or one or more masked with the point cloud of the model, or the model after adjustment, or the model after registration; the display module may display one or more of the model, the point cloud, the other imagery than the point cloud, the one or more of fusions and the one or more masked with the point cloud, wherein change in shape, structure and position of the body part is highlighted to facilitate diagnosis by medical staff.

The processing module may obtain a 3D data model of the body part, and set brightness or hue of a light component of at least one voxel of the model with reference to one or more of a spatial distribution and spectral characteristics of illuminance of light source of a camera of the data acquisition module, a spectral characteristics of a tissue of the body part, and a relative position between the light source, the camera and the body part, wherein a difference between H value of a hue of the light component of a first voxel of a tissue and H value of a hue of the light component of a second voxel of the tissue is less than a first threshold.

The processing module may validate or modify brightness of a light component of a voxel of the model by referencing a correlation between brightness of a light component of a voxel of the model and brightness of the light component of a point structure of the point cloud, wherein coordinates of the voxel and coordinates of the point structure are identical or correlated, and/or to validate or modify a hue of the light component of the voxel causing a difference between a H value in HSV color space of the hue of the light component of the voxel and a H value of a hue of the light component of the point structure to be less than a second threshold, wherein the light component of the voxel and of the point structure is representative of a light spot of anatomy of the body part. The processing module may further validate or modify brightness or hue of the light component of at least one voxel in a neighborhood of the voxel with reference to one or more of the brightness and the H value of the light component of the voxel, a spatial distribution and spectral characteristics of illuminance of a light source of a camera of the data acquisition module, a spectral characteristics of a tissue of the body part, and a relative position between the light source, the camera and the body part, wherein a difference between H value of a hue of the light component of a first voxel of the tissue and H value of a hue of the light component of a second voxel of the tissue is less than the first threshold.

The processing module may perform a matching of the point cloud with the adjusted model to validate or modify the adjusted model or obtain a position of the body part. The processing module may further perform, referencing the other imagery including one or more of CT, MRI, and ultrasound, one or more of the following: conducting a second registration of the model, comprising validating or modifying data of the model or obtaining a position of the body part, and controlling the robot or the endoscope by referencing the second registration.

The processing module may obtain a data set of features of or markings on one or more of the point cloud, the model and the other imagery and further perform one or more of the following: conducting a third registration of the adjusted model with reference to the data set, including validating or modifying the model or obtaining a position of the body part; controlling the surgical robot or the endoscope with reference to the third registration; obtaining data of fusion of the data set with one or more of the point cloud, the model and the other imagery, or data of masking one or more of the point cloud, the model and the other imagery with the data set; wherein the display module may display the data.

The processing module may set a mask on a light component of a first voxel of the model, and/or a light component of a second voxel of the model after the adjustment and/or a light component of a third voxel of the model after the registration, wherein coordinates of the first, second and third voxels are correlated with or being identical to coordinates of a respective point structure of the point cloud.

The data acquisition module may obtain a point cloud and imagery other than the point cloud corresponding a serial number of n+1, wherein the processing module may register the model with reference to the point cloud, the imagery corresponding the serial number of n+1 and a registered model corresponding to a serial number of n.

The invention discloses an image processing method in accordance with the above system comprising the steps of:

Step1: obtaining by a data acquisition module a model of three-dimensional spectral data, or the model and a point cloud, or the model, the point cloud, and other imagery than the point cloud of a body part;

Step2: performing by a data processing module one or more of the following: 3D printing the model; adjusting the model referencing the point cloud; 3D printing the adjusted model; performing a first registration of the adjusted model with reference to the point cloud; 3D printing the registered model; controlling a surgical robot or an endoscope with reference to the first registration; obtaining one or more of fusions of two or more of the model, the point cloud, and the other imagery than the point cloud or one or more masked with the point cloud of the model, or the model after adjustment, or the model after registration;

or displaying by a display module one or more of the model, the point cloud, the other imagery than the point cloud, the one or more of fusions and the one or more masked with the point cloud, wherein change in shape, structure and position of the body part is highlighted to facilitate diagnosis by medical staff.

According to the method, the obtaining the model of three-dimensional spectral data may comprise the steps of:

acquiring a 3D data model of the body part;

setting brightness or hue of a light component of at least one voxel of the model with reference to one or more of a spatial distribution and spectral characteristics of illuminance of light source of a camera of the data acquisition module, a spectral characteristics of a tissue of the body part, and a relative position between the light source, the camera and the body part, wherein a difference between H value of a hue of the light component of a first voxel of a tissue and H value of a hue of the light component of a second voxel of the tissue is less than a first threshold.

According to the method, the adjusting the model may comprise the steps of:

validating or modifying a first brightness of a light component of a voxel of the model by referencing a correlation between brightness of a light component of a voxel of the model and brightness of the light component of a point structure of the point cloud, wherein coordinates of the voxel and coordinates of the point structure are identical or correlated, and/or validating or modifying a hue of the light component of the voxel causing a difference between a H value in HSV color space of the hue of the light component of the voxel and a H value of a hue of the light component of the point structure to be less than a second threshold, wherein data of the light component of the voxel and of the point structure is representative of a light spot of anatomy of the body part; validating or modifying brightness or hue of the light component of at least one voxel in a neighborhood of the voxel with reference to one or more of the brightness and the H value of the light component of the voxel, a spatial distribution and spectral characteristics of illuminance of a light source of a camera of the data acquisition module, a spectral characteristics of a tissue of the body part, and a relative position between the light source, the camera and the body part, wherein a difference between H value of a hue of the light component of a first voxel of the tissue and H value of a hue of the light component of a second voxel of the tissue is less than the first threshold. According to the method, the first registration may comprise performing a matching of the point cloud with the adjusted model to validate or modify the adjusted model or to obtain a position of the body part.

The method may further comprise the steps of:

conducting a second registration of the model, comprising validating or modifying data of the model or obtaining a position of the body part referencing the other imagery including one or more of CT, MRI, and ultrasound;

controlling the robot or the endoscope by referencing the second registration.

The method may further comprise the steps of:

Step 1: obtaining a data set of features of or markings on one or more of the point cloud, the model and the other imagery;

Step2: performing one or more of the following: conducting a third registration of the adjusted model with reference to the data set, including validating or modifying the model or obtaining a position of the body part; controlling the surgical robot or the endoscope with reference to the third registration; obtaining data of fusion of the data set with one or more of the point cloud, the model and the other imagery, or data of masking one or more of the point cloud, the model and the other imagery with the data set; displaying the data.

According to the method, the masking may comprise the steps of:

setting a mask on a light component of a first voxel of the model, and/or a light component of a second voxel of the model after the adjustment and/or a light component of a third voxel of the model after the registration, wherein coordinates of the first, second and third voxels are correlated with or being identical to coordinates of a respective point structure of the point cloud.

The method may further comprise the steps of: obtaining a point cloud and imagery other than the point cloud corresponding a serial number of n+1;

registering the model with reference to the point cloud, the imagery corresponding to the serial number of n+1 and a registered model corresponding to a serial number of n. The invention is promising to improve positioning of anatomy of a body part in an endoscopy or a robotic surgery and thereby to provide a more accurate navigation of a surgical robot. In addition, the enhanced display of masked fusion may provide an intuitive view to changes of the body part in real time.

### BRIEF DESCRIPTION OF THE DRAWINGS

FIG. 1 is an illustration of the system architecture.

FIG. 2 is an illustration of modular construction of the data acquisition module.

FIG. 3 is an illustration of modular construction of the display module.

FIG. 4 is an illustration of modular construction of the processing module.

FIG. 5 is an illustration of system outline.

FIG. 6 is the schematics for system operation.

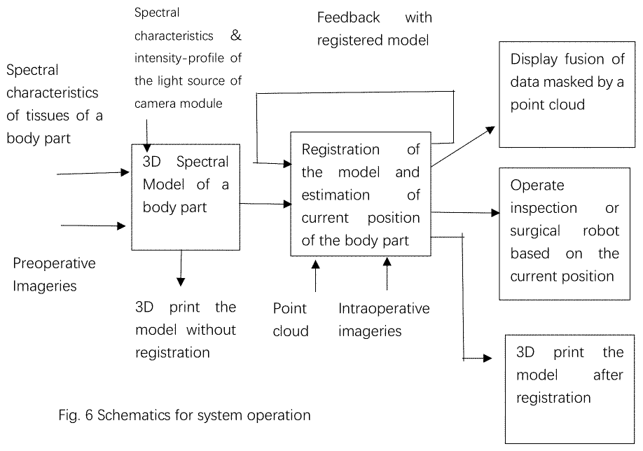

### DETAILED DESCRIPTION

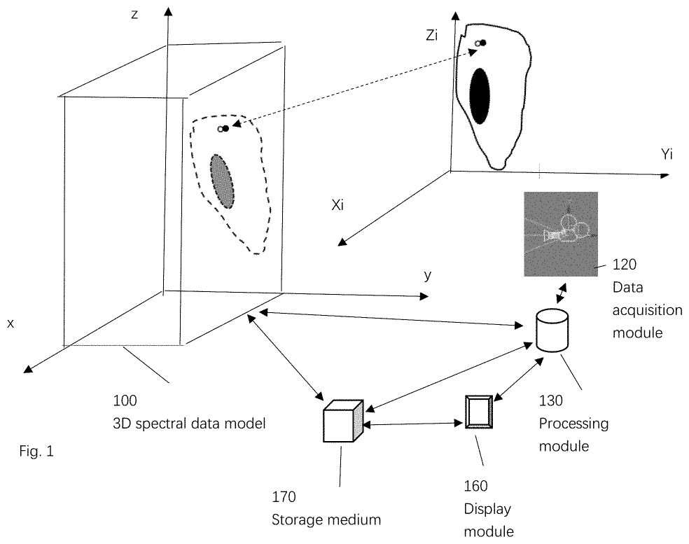

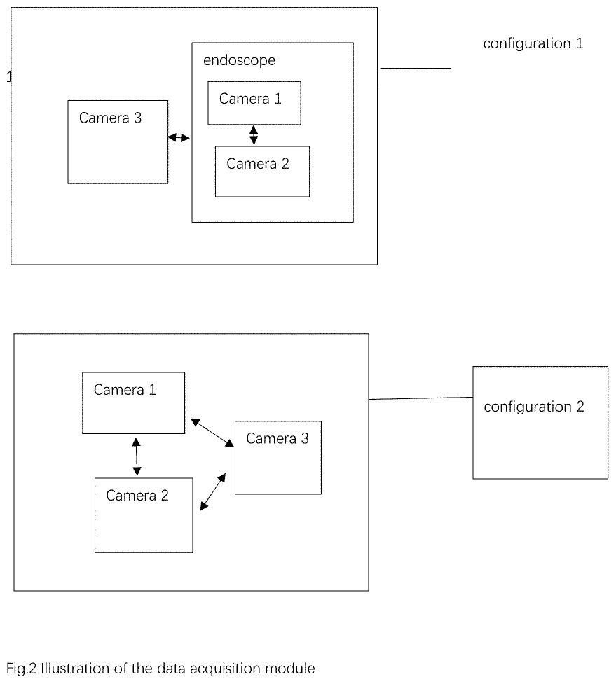

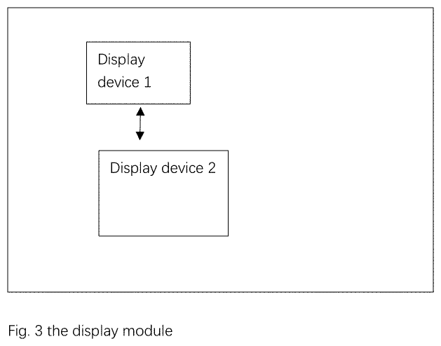

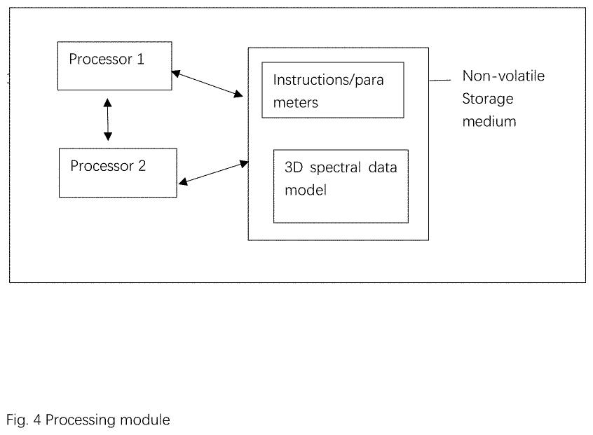

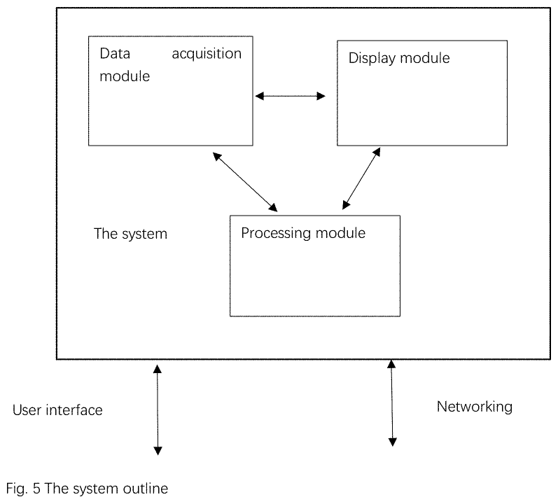

The following example embodiments are provided to illustrate the present invention without limiting the scope of the invention. The system and method disclosed in the present invention relate to the following observation and rational: The 3D imageries obtained preoperatively may not reflect the actual position of the body part in an endoscopy or a surgery. Although data of intraoperative radioactive imageries may help with dynamic positioning, their acquisition may invoke risks to the safety of patient and medical staffs as well and demand additional complexities to the operation room set up. Alternatively, or complementarily, image data or point cloud captured by one or more cameras of an endoscope may contain pertinent real-time information of anatomy of the body part at the surgical site that may be used as an expedite reference for registering a data model of a body part or other imageries for positioning the body part. The point cloud comprises a collection of point structures of a surface in a 3D space in an array of the point structures each comprising coordinates of a point structure and R, G, B color component values of the point structure. As shown in FIG. 1-FIG. 5, the data acquisition module may comprise one or more cameras or one or more endoscopes having one or more cameras; the processing module may include one or more processors and connected non-volatile storage medium having instructions and parameters stored thereupon to be accessed by the one or more processors to run one or more programs for endoscopy or robotic surgery. The display module may comprise one or more display devices. The data acquisition module, the processing module and the display module may be fully or partially integrated in one apparatus or be coupled by communication link. The system may include user interfaces and be networked externally for control and data. The XᵢYᵢZᵢ coordinate system may represent a coordinate system of the model during preoperative planning, while the xyz coordinate system may represent the intraoperative coordinate system wherein data are mapped from the models in XᵢYᵢZᵢ coordinate system. The smallest dots in FIG. 1 are representative of voxels of a model of a human body part, the ellipses representative of features of the body part and the largest contour presentative of the boundary of the body part. A human body part may be simulated as a model of three-dimensional data structure set in a coordinate system, wherein differences between individuals may manifest as anisotropic smooth expansion or compression, displacement or rotation in each of the dimensions.

The 3D spectral data model of a body part may preferably be represented by M(x, y, z, λᵢ, n), wherein M is representative of a voxel of the model; x, y, z are representative of the coordinates of the voxel in a coordinate system; λᵢ is representative of a light component of the voxel, wherein for example λ₁=(R, G, B) is representative of a day light component; λ₂=(r, g, b) is representative of a fluorescence light component; λ₃=(ρ_{c}) is representative of a light component corresponding to a CT image value; λ₄=(ρₘ) is representative of a light component corresponding to a MRI image value; λ₅=(ρₛ) is representative of a light component corresponding to a ultrasound image value; λ₆ is representative of a light component corresponding to a mask value and etc.; n is representative of a time sequence number in one instance of application. In practical application, a voxel may comprise one or more of the above λᵢ values, or other metric values not listed above.

Similarly, a point cloud of a surface of anatomy of the body part may be preferably represented by P (x, y, z, Lⱼ, n), wherein P is representative of a point structure; x, y, z are representative of the coordinates of the point structure in the coordinate system; Lⱼ is representative of values of the point structure in a color space, and j is representative of the spectrum of light, for example j=1 for day light, and j=2 for fluorescent light; n is representative of a time sequence number in one instance of application.

A first step in building a model of a body part may comprise extracting, from preoperative 3D imageries of CT, MRI and ultrasonics, a 3D data set representative of morphological structures of anatomy of tissues of the body part, wherein the tissues may include for example skin, mucous membranes, fat, nerves, fascia, muscles, blood vessels, internal organs, bones and etc. The next step may comprise setting brightness and hue values for each voxel of the data set and thereby fill the model with spectral data. Take an example of the process of building the day light component of the model assuming the light source of the camera of the endoscope is of the day light spectrum. Since a tissue of a body part may exhibit consistent spectral characteristics with respect to illumination by the day light, tissues classifiable for example by a CT or MM image data may be set a generic color, which may be characterized through a spectral analysis of the issue illuminated by a source of the day light, wherein a difference between H value of a hue of a light component of a first voxel of a tissue and H value of a hue of the light component of a second voxel of the tissue may be set to be less than a first threshold. Since the intensity of light wave attenuates as it propagates in space, the profile of the attenuation of the illumination of a light source of a camera of the data acquisition module of the system may be calibrated or simulated into one or more look up tables with respect to a view point of the camera and a relative position between the light source, the cameras and the body part, in a defined space of an operation room. Alternatively, values of a light component of a voxel of the model may be calibrated by referencing correlated values of the light component of a correlated point structure of a point cloud of the body part comprising the following steps:

Setting brightness of a day light component of a voxel of the model by referencing a correlation between the brightness and a brightness of the light component of a point structure of the point cloud, wherein coordinates of the voxel and coordinates of the point structure of the point cloud are identical or correlated, wherein both the voxel and the point structure refer a light spot at the surface of anatomy of the body part;

Setting a hue of the day light component of the voxel causing a difference between a H value in HSV color space of the hue of the day light component of the voxel and a H value of a hue of the day light component of the point structure of the point cloud to be less than a first threshold;

Setting brightness or hue of the day light component of at least one voxel in the neighborhood of the voxel with reference to one or more of the brightness and the hue of the day light component of the voxel, a spatial distribution and spectral characteristics of illuminance of light source of a camera of the data acquisition module, a spectral characteristics of a tissue of the body part, and a relative position between the light source, the camera and the body part, wherein a difference between H value of a hue of a light component of a first voxel of the tissue and H value of a hue of the light component of a second voxel of the tissue is less than a second threshold, wherein the day light component of the voxel and the point structure is representative of a day light spot of a surface of anatomy of the body part.

A pair of binocular vision cameras may capture image data of the body part under the illumination of the light source and depth data and a point cloud coupled with the image data may be extracted. Alternatively, a first camera may capture an image of a surface of anatomy of the body part while a second camera may capture coordinates of point structures of a point cloud; Alternatively, a camera with a 3D sensor may capture coordinates and values of point structures of a point cloud in a single shot. The sampling rate of voxels and the spatial resolution of the model conform to the Nyquist sampling theorem.

A built model of a body part may be adjusted in real time in an endoscopy or a robotic surgery in synchronization with each updated acquisition of the point cloud of the body part, wherein the data processing module may receive or retrieve a model and validate or modify the model through a similar process described above.

An example for registration may comprise the steps of: obtaining a point cloud including a body part; extracting boundary of the body part in the point cloud automatically by an algorithm, or by manual marking, or by both means above; obtaining an adjusted model of the body part by a process described above; performing a matching algorithm of least mean square error between the adjusted model and the point cloud within the boundary in the steps of: Step 1, locating voxels with coordinates corresponding to coordinates of point structures of the point cloud; Step 2, calculating a mean square error between the values of the light component of the voxels and of the corresponding point structures of the point cloud; Step 3, obtaining new coordinates of the voxels after transformation of the initial coordinates comprising one or more of translation, rotation, and scaling; Step 4, calculating the least mean square error between values of the light component of the voxels with the new coordinates and values of the light component of the point structures of the point cloud; step 5, repeating steps 3-4 by traversing parameters and obtaining a set of parameters comprising data of displacement, rotation, and scaling. The registration may be completed by a coordinate transform based on the set of parameters, or by position variable transforms with respect to individual voxels. The registered model or a current position of the body part estimated through the registration of the model may be used to guide a user operating an endoscope or to navigate a surgical robot.

One preferred presentation of the model may be set a mask to a light component of a first voxel of the model, and/or a light component of a second voxel of the model after the adjustment and/or a light component of a third voxel of the model after the registration, wherein coordinates of the first, second and third voxels are correlated with or being identical to coordinates of a respective point structure of the point cloud, wherein the mask value may comprise a value of a light component of the respective point structure of the point cloud.

An optional recursive Kalman filtering with the registered model may be used as auxiliary data assisting registering the model with newly acquired point cloud or other imagery during endoscopy or surgery.

In an endoscopic surgery, an outlier of anatomy of the body part at surgical site is normally exposed in the view field first and the hierarchical structure of the anatomy is gradually developed as the operation proceeds, wherein the amount of information obtained by the endoscope is cumulatively increased. Either a surgeon or a surgical robot has to start with the limited information. Alternatively, a machine learning based registration may be developed as an emulation of a surgeon exercising his or her surgical experiences. The surgical robot may learn not only from its own practices, but also directly or indirectly from practices of its companion robots as well, and therefore excel the surgeon in speed and accuracy of the learning. An external 3D printing apparatus may be configured to be connected to the processing module and perform 3D printing of the model.

1. An image processing system, comprising a data acquisition module, a processing module and a display module; the data acquisition module is configured to obtain a model of three-dimensional spectral data, or the model and a point cloud, or the model, the point cloud, and other imagery than the point cloud of a body part; the processing module is configured to perform one or more of the following: 3D printing the model; adjusting the model referencing the point cloud; 3D printing the adjusted model; performing a first registration of the adjusted model with reference to the point cloud; 3D printing the registered model; controlling a surgical robot or an endoscope with reference to the first registration; obtaining one or more of fusions of two or more of the model, the point cloud, and the other imagery than the point cloud or one or more masked with the point cloud of the model, or the model after adjustment, or the model after registration; the display module is configured to display one or more of the model, the point cloud, the other imagery than the point cloud, the one or more of fusions and the one or more masked with the point cloud, wherein change in shape, structure and position of the body part is highlighted to facilitate diagnosis by medical staff.

2. The system of claim 1, wherein the processing module is configured to obtain a 3D data model of the body part, and set brightness or hue of a light component of at least one voxel of the model with reference to one or more of a spatial distribution and spectral characteristics of illuminance of light source of a camera of the data acquisition module, a spectral characteristics of a tissue of the body part, and a relative position between the light source, the camera and the body part, wherein a difference between H value of a hue of the light component of a first voxel of the tissue and H value of a hue of the light component of a second voxel of the tissue is less than a first threshold.

3. The system of claim 1, wherein the processing module is configured to validate or modify brightness of a light component of a voxel of the model by referencing a correlation between the brightness of the light component of the voxel of the model and brightness of the light component of a point structure of the point cloud, wherein coordinates of the voxel and coordinates of the point structure are identical or correlated, and/or to validate or modify a hue of the light component of the voxel causing a difference between a H value in HSV color space of the hue of the light component of the voxel and a H value of a hue of the light component of the point structure to be less than a second threshold; the processing module is further configured to validate or modify brightness or hue of the light component of at least one voxel in a neighborhood of the voxel with reference to one or more of the brightness and the H value of the light component of the voxel, a spatial distribution and spectral characteristics of illuminance of a light source of a camera of the data acquisition module, a spectral characteristics of a tissue of the body part, and a relative position between the light source, the camera and the body part, wherein a difference between H value of a hue of the light component of a first voxel of the tissue and H value of a hue of the light component of a second voxel of the tissue is less than the first threshold.

4. The system of claim 2, wherein the light component of the voxel and of the point structure is representative of a light spot of anatomy of the body part.

5. The system of claim 1, wherein the processing module is further configured to perform a matching of the point cloud with the adjusted model to validate or modify the adjusted model or obtain a position of the body part.

6. The system of claim 1, the processing module is further configured to, referencing the other imagery including one or more of CT, MM, and ultrasound, perform one or more of the following: conducting a second registration of the model or the adjusted model, comprising validating or modifying data of the model or the adjusted model, or obtaining a position of the body part; controlling the robot or the endoscope by referencing the second registration.

7. The system of claim 1, wherein the processing module is configured to obtain a data set of features or markings on one or more of the point cloud, the model and the other imagery; further perform one or more of the following: conducting a third registration of the model or the adjusted model with reference to the data set, including validating or modifying the model or the adjusted model, or obtaining a position of the body part; controlling the surgical robot or the endoscope with reference to the third registration; obtaining fusion of the data set with one or more of the point cloud, the model, the adjusted model, the registered model and the other imagery, or masking, with the data set, one or more of the point cloud, the model, the adjusted model, the registered model and the other imagery; the display module is configured to display the fusion or the masking.
The system of claim 1, wherein the processing module is further configured to set a mask on a light component of a first voxel of the model, and/or a light component of a second voxel of the model after the adjustment and/or a light component of a third voxel of the model after the registration, wherein coordinates of the first, second and third voxels are correlated with or being identical to coordinates of a respective point structure of the point cloud.

8. The system of claim 8, wherein the mask comprises being correlated with a value of a light component of the respective point structure of the point cloud.

9. The system of claim 1, the data acquisition module is further configured to obtain a point cloud and imagery other than the point cloud corresponding a serial number of n+1, the processing module is further configured to register the model with reference to the point cloud, the imagery corresponding the serial number of n+1 and a registered model corresponding to a serial number of n.

10. A method of image processing, comprising the steps of:
Step1: obtaining by a data acquisition module a model of three-dimensional spectral data, or the model and a point cloud, or the model, the point cloud, and other imagery than the point cloud of a body part;
Step2: performing by a data processing module one or more of the following:
3D printing the model; adjusting the model referencing the point cloud; 3D printing the adjusted model; performing a first registration of the adjusted model with reference to the point cloud; 3D printing the adjusted model; performing a first registration of the adjusted model with reference to the point cloud; 3D printing the registered model; controlling a surgical robot or an endoscope with reference to the first registration; obtaining one or more of fusions of two or more of the model, the point cloud, and the other imagery than the point cloud or one or more masked with the point cloud of the model, or the model after adjustment, or the model after registration;
or displaying by a display module one or more of the model, the point cloud, the other imagery than the point cloud, the one or more of fusions and the one or more masked with the point cloud, wherein change in shape, structure and position of the body part is highlighted to facilitate diagnosis by medical staff.

11. The method of claim 11, wherein the obtaining the model of three-dimensional spectral data comprising the steps of:
acquiring a 3D data model of the body part;
setting brightness or hue of a light component of at least one voxel of the model with reference to one or more of a spatial distribution and spectral characteristics of illuminance of light source of a camera of the data acquisition module, a spectral characteristics of a tissue of the body part, and a relative position between the light source, the camera and the body part, wherein a difference between H value of a hue of the light component of a first voxel of a tissue and H value of a hue of the light component of a second voxel of the tissue is less than a first threshold.

12. The method of claim 11, wherein the adjusting the model comprising the steps of:
validating or modifying a first brightness of a light component of a voxel of the model by referencing a correlation between brightness of a light component of a voxel of the model and brightness of the light component of a point structure of the point cloud, wherein coordinates of the voxel and coordinates of the point structure are identical or correlated, or to set or change a hue of the light component of the voxel causing a difference between a H value in HSV color space of the hue of the light component of the voxel and a H value of a hue of the light component of the point structure to be less than a second threshold; validating or modifying brightness or hue of the light component of at least one voxel in a neighborhood of the voxel with reference to one or more of the brightness and the H value of the light component of the voxel, a spatial distribution and spectral characteristics of illuminance of a light source of a camera of the data acquisition module, a spectral characteristics of a tissue of the body part, and a relative position between the light source, the camera and the body part, wherein a difference between H value of a hue of the light component of a first voxel of the tissue and H value of a hue of the light component of a second voxel of the tissue is less than the first threshold.

13. The method of claim 12, wherein data of the light component of the voxel and of the point structure is representative of a light spot of anatomy of the body part.

14. The method of claim 11, wherein the first registration comprising performing a matching of the point cloud with the adjusted model to validate or modify the adjusted model or to obtain a position of the body part.

15. The method of claim 11, further comprising the steps of:
conducting a second registration of the model, comprising validating or modifying data of the model or obtaining a position of the body part referencing the other imagery including one or more of CT, MM, and ultrasound;
controlling the robot or the endoscope by referencing the second registration.

16. The method of claim 11, further comprising the steps of:
Step1: obtaining a data set of features of or markings on one or more of the point cloud, the model and the other imagery;
Step2: performing one or more of the following: conducting a third registration of the adjusted model with reference to the data set, including validating or modifying the model or obtaining a position of the body part; controlling the surgical robot or the endoscope with reference to the third registration; obtaining data of fusion of the data set with one or more of the point cloud, the model and the other imagery, or data of masking one or more of the point cloud, the model and the other imagery with the data set; displaying the data.

17. The method of claim 11, wherein the masking comprising the steps of:
setting a mask on a light component of a first voxel of the model, and/or a light component of a second voxel of the model after the adjustment and/or a light component of a third voxel of the model after the registration, wherein coordinates of the first, second and third voxels are correlated with or being identical to coordinates of a respective point structure of the point cloud.

18. The method of claim 18, wherein the mask comprises being correlated with a value of the point structure.

19. The method of claim 11, further comprising the steps of: obtaining a point cloud and imagery other than the point cloud corresponding a serial number of n+1;
registering the model with reference to the point cloud, the imagery corresponding to the serial number of n+1 and a registered model corresponding to a serial number of n.
